(19) United States
(12) Patent Application Publication
(10) Pub. No.: US 2012/0095508 A1
(43) Pub. Date: Apr. 19, 2012

(54) Portable and hand-held device for pulling thoracic vertebrates of a user upwardly and forwardly into vertical alignment

(76) Inventor: Susan Salnick, Rivervale, NJ (US)

(21) Appl. No.: 13/317,204

(22) Filed: Oct. 12, 2011

Related U.S. Application Data
(60) Provisional application No. 61/404,967, filed on Oct. 13, 2010.

(51) Int. Cl. A61F 5/00

(52) U.S. Cl. 606/237

(57) ABSTRACT

A portable and hand-held device that generally fits like a vest, and which pulls thoracic vertebrates of a user upwardly and forwardly into vertical alignment. The device includes a continuous loop. The continuous loop is flexible, and has memory so as to allow the continuous loop to stay bent in any desired position to pull the thoracic vertebrates of the user upwardly and forwardly into the vertical alignment.

### 1. CROSS REFERENCE TO RELATED APPLICATIONS

The instant non-provisional patent application claims priority from provisional patent application No. 61/404,967, filed Oct. 13, 2010, entitled PORTABLE AND HAND-HELD DEVICE FOR PULLING THORACIC VERTEBRATES OF A USER UPWARDLY AND FORWARDLY INTO VERTICAL ALIGNMENT, and incorporated herein by reference thereto.

### 2. BACKGROUND OF THE INVENTION

A. Field of the Invention

The embodiments of the present invention relate to a device for pulling thoracic vertebrates of a user upwardly and forwardly into vertical alignment, and more particularly, the embodiments of the present invention relate to a portable and hand-held device for pulling thoracic vertebrates of a user upwardly and forwardly into vertical alignment.

B. Description of the Prior Art

Numerous innovations for vertebra alignment devices have been provided in the prior art. Even though these innovations may be suitable for the specific individual purposes to which they address, nevertheless, they differ from the embodiments of the present invention in that they do not teach a portable and hand-held device for pulling thoracic vertebrates of a user upwardly and forwardly into vertical alignment.

### 3. SUMMARY OF THE INVENTION

Thus, an object of the embodiments of the present invention is to provide a portable and hand-held device for pulling thoracic vertebrates of a user upwardly and forwardly into vertical alignment, which avoids the disadvantages of the prior art.

Briefly stated, another object of the embodiments of the present invention is to provide a portable and hand-held device that generally fits like a vest, and which pulls thoracic vertebrates of a user upwardly and forwardly into vertical alignment. The device includes a continuous loop. The continuous loop is flexible, and has memory so as to allow the continuous loop to stay bent in any desired position to pull the thoracic vertebrates of the user upwardly and forwardly into the vertical alignment.

The novel features considered characteristic of the embodiments of the present invention are set forth in the appended claims. The embodiments of the present invention themselves, however, both as to its construction and to its method of operation together with additional objects and advantages thereof will be best understood from the following description of the embodiments of the present invention when read and understood in connection with the accompanying figures of the drawing.

### 4. BRIEF DESCRIPTION OF THE FIGURES OF THE DRAWING

FIG. 1 is a diagrammatic perspective view of the portable and hand-held device of the embodiments of the present invention pulling thoracic vertebrates of a user upwardly;

FIG. 2 is a diagrammatic perspective view of the portable and hand-held device of the embodiments of the present invention pulling thoracic vertebrates of a user forwardly; and

FIG. 3 is an enlarged diagrammatic perspective view of the portable and hand-held device of the embodiments of the present invention identified by ARROW 3 in FIGS. 1 and 2.

### 5. LIST OF REFERENCE NUMERALS UTILIZED IN THE FIGURES OF THE DRAWING

### A. General.

10 portable and hand-held device of embodiments of present invention for pulling thoracic vertebrates 12 of user 14 upwardly and forwardly into vertical alignment
12 thoracic vertebrates of user 14
14 user

### B. Configuration of Portable and Hand-Held Device 10.

16 continuous loop
18 core of continuous loop 16
20 sheath of continuous loop 16
22 wire of core 18 of continuous loop 16
24 handles of continuous loop 16
25 hands of user 14
26 intermediate portion of continuous loop 16
28 thorax of user 14
30 shoulder blades of user 14
32 arms of user 14

### 6. DETAILED DESCRIPTION OF THE PREFERRED EMBODIMENTS

### A. General.

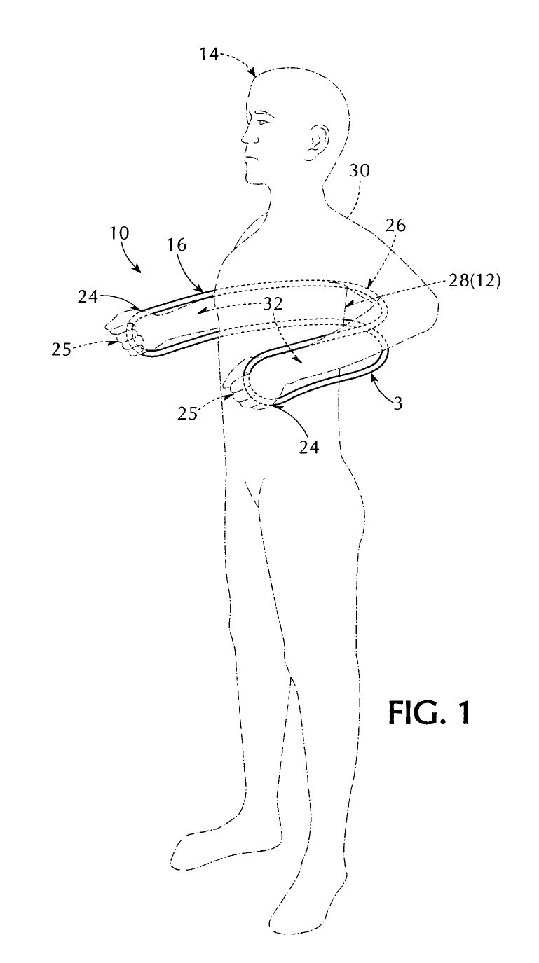

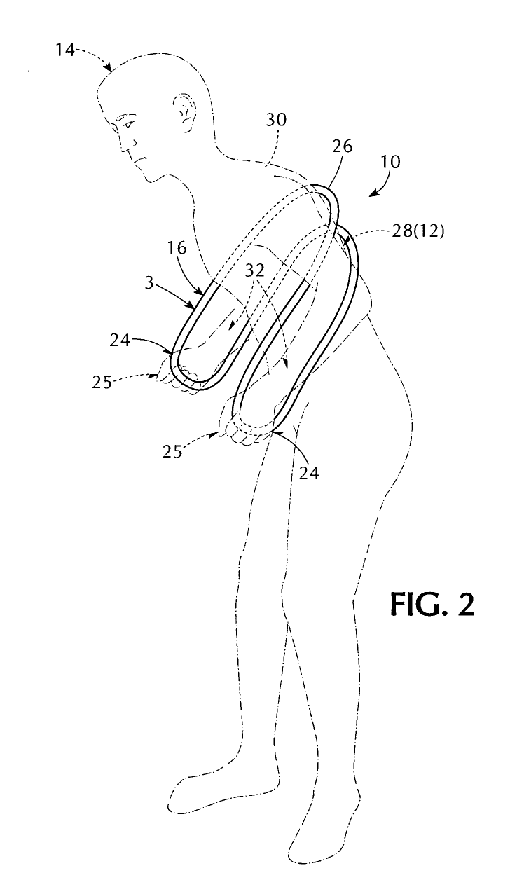

Referring now to FIGS. 1 and 2, which are, respectively, a diagrammatic perspective view of the portable and hand-held device of the embodiments of the present invention pulling thoracic vertebrates of a user upwardly, and a diagrammatic perspective view of the portable and hand-held device of the embodiments of the present invention pulling thoracic vertebrates of a user forwardly, the portable and hand-held device of the embodiments of the present invention is shown generally at 10 for pulling thoracic vertebrates 12 of a user 14 upwardly and forwardly into vertical alignment.

### B. Configuration of the Portable and Hand-Held Device 10.

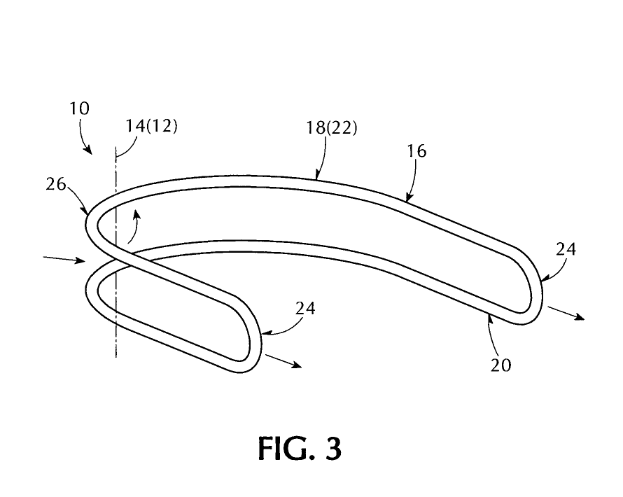

The configuration of the portable and hand-held device 10 can best be seen in FIG. 3, which is an enlarged diagrammatic perspective view of the portable and hand-held device of the embodiments of the present invention identified by ARROW 3 in FIGS. 1 and 2, and as such, will be discussed with reference thereto.

The portable and hand-held device 10 comprises a continuous loop 16.

The continuous loop 16 is flexible, but without memory so as to allow the continuous loop 16 to stay bent in any desired position.

The continuous loop 16 comprises a core 18 and a sheath 20.

The sheath 20 of the continuous loop 16 encases the core 18 of the continuous loop 16.

The core 18 of the continuous loop 16 is flexible, but without memory so as to allow the continuous loop 16 to stay bent in any desired position, and can be a wire 22, but is not limited to that.

The sheath 20 of the continuous loop 16 is flexible, is soft for comfort for the user 14, and can be injection molded around the core 18 of the continuous loop 16, but is not limited to that.

The continuous loop 16 is generally elliptical prior to use by the user 14, and folds over itself to form handles 24.

The handles 24 of the continuous loop 16 project forwardly from each side of the user 14, and are engaged by the hands 25 of the user 14 for being pushed forwardly during use by the user 14, while an intermediate portion 26 of the continuous loop 16 between the handles 24 of the continuous loop 16 goes around the thorax 28 of the user 14 during use by the user 14 (FIGS. 1 and 2).

When the hands 25 of the user 14 are pushed forwardly, thereby pushing forwardly the handles 24 of the continuous loop 16, the vertebra 12 of the user 14 are pulled forwardly to thereby be vertically aligned, while the upper thorax 28 of the user 14 is lifted, the shoulder blades 30 of the user 14 are drawn toward each other, and the arms 32 of the user 14 are strengthened (FIGS. 1 and 2).

The portable and hand-held device 10 can also be used by an attendant to lift the user 14 to a sitting position or from a horizontal position because the portable and hand-held device 10 is easily bendable to lay flat. When not in use, the portable and hand-held device 10 can be wrapped around the user 14 whereby it can be worn like a vest.

### C. Impressions.

It will be understood that each of the elements described above, or two or more together, may also find a useful application in other types of constructions differing from the types described above.

While the embodiments of the present invention have been illustrated and described as embodied in a portable and hand-held device for pulling thoracic vertebrates of a user upwardly and forwardly into vertical alignment, however, it is not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt it for various applications without omitting features that from the standpoint of prior art fairly constitute characteristics of the generic or specific aspects of the embodiments of the present invention.

The invention claimed is:

1. A portable and hand-held device for pulling thoracic vertebrates of a user upwardly and forwardly into vertical alignment, comprising:
a continuous loop;
wherein said continuous loop is flexible; and
wherein said continuous loop has memory so as to allow said continuous loop to stay bent in any desired position for pulling the thoracic vertebrates of the user upwardly and forwardly into the vertical alignment.

2. The device of claim 1, wherein said continuous loop comprises:
a) a core; and
b) a sheath;
wherein said sheath of said continuous loop encases said core of said continuous loop.

3. The device of claim 2, wherein said core of said continuous loop is flexible.

4. The device of claim 2, wherein said core of said continuous loop has memory so as to allow said continuous loop to stay bent in the any desired position for pulling the thoracic vertebrates of the user upwardly and forwardly into the vertical alignment.

5. The device of claim 2, wherein said core of said continuous loop is made of wire.

6. The device of claim 2, wherein said sheath of said continuous loop is flexible; and
wherein said sheath of said continuous loop is soft for comfort for the user.

7. The device of claim 2, wherein said sheath of said continuous loop is injection molded around said core of said continuous loop.

8. The device of claim 1, wherein said continuous loop is generally elliptical prior to use by the user.

9. The device of claim 1, wherein said continuous loop folds over itself to form handles;
wherein said handles of said continuous loop project forwardly from each side of the user;
wherein said handles of said continuous loop are engaged by the hands of the user for being pushed forwardly during use by the user, while an intermediate portion of said continuous loop between said handles of said continuous loop goes around the thorax of the user during use by the user; and
wherein when the hands of the user are pushed forward, thereby pushing forward said handles of said continuous loop, the vertebra of the user are pulled forwardly to thereby be vertically aligned, while the upper thorax of the user is lifted, the shoulder blades of the user are drawn toward each other, and the arms of the user are strengthened.
